(19) United States
(12) Patent Application Publication
(10) Pub. No.: US 2009/0215727 A1
(43) Pub. Date: Aug. 27, 2009

(54) INHIBITION OF ATP-MEDIATED, P2X7 DEPENDENT PATHWAYS BY PYRIDOXAL-5-PHOSPHATE AND VITAMIN B6 RELATED COMPOUNDS

(75) Inventor: Deborah Douglas, Winnipeg (CA)

Correspondence Address: MERCHANT & GOULD PC, P.O. BOX 2903, MINNEAPOLIS, MN, 55402-0903, US

(73) Assignee: MEDICURE INTERNATIONAL INC., HOLETOWN, CA (US)

(21) Appl. No.: 11/913,654

(22) PCT Filed: May 5, 2006
(86) PCT No.: PCT/CA06/00717

§ 371 Date: May 21, 2008

Related U.S. Application Data
(60) Provisional application No. 60/677,830, filed on May 5, 2005.

(51) Int. Cl. A61K 31/675; A61P 29/00

(52) U.S. Cl. 514/89

(57) ABSTRACT

P5P can be used as effective treatments for the modulation of P2X7, IL-1β, and inflammation response, and for diseases in which prevention of P2X7-dependent pathways or prevention of release of IL-1β is desirable, such as epithelial cancer, leukemia, brain tumors, spinal cord injury, tuberculosis, Alzheimer's Disease, neurodegenerative diseases, autosomal recessive polycystic kidney disease, diabetes, including type I diabetes, prostate cancer, and osteoporosis, bone formation and resorption.

### CROSS REFERENCE

This patent claims priority to U.S. provisional patent application 60/677,830, filed on May 5, 2005, which is incorporated herein in its entirety.

### BACKGROUND

Increasing evidence suggests that substantial amounts of ATP can accumulate in the extra-cellular space under a variety of physiological and pathophysiological conditions (Vassort, 2001, Harden et al, 1994, Dubyak et al, 1993). ATP acts on cells via P2-purinergic receptors to trigger a number of different responses including secretion, chemotaxis, proliferation, transcription factor activation and cytotoxicity (Di Virigilio et al, 2001). In addition, ATP can also be a powerful pro-apoptotic agent mediating its effects through the specific activation of P2X7 receptors (Apasov et al, 1995, Zoeteweij et al, 1996), one of seven known P2-purinergic receptors. When ATP binds to P2X7 receptors it facilitates a rapid bi-directional flux of cations thereby triggering depolarization, collapse of the Na+ and K+ gradients, and massive influx of Ca2+. Furthermore, continued stimulation of P2X7 receptors causes formation of large, nonspecific pores, allowing permeability of molecules up to 800 Da via recruitment of a distinct pore-forming moiety (Zanovello et al, 1990, Zheng et al, 1991, Surprenant et al, 1996).

Vitamin B₆ is an essential nutrient for human health. A metabolically active form of the vitamin is pyridoxal-5′-phosphate (P5P) and it is involved as a cofactor in many enzymatically controlled reactions including amino acid metabolism, glucose metabolism, heme synthesis, phospholipid synthesis and neurotransmitter synthesis. Several dietary compounds can function as a precursor to P5P. These include pyridoxine, pyridoxal, pyridoxamine as well as P5P itself. The term vitamin B₆ usually refers to pyridoxine hydrochloride as this is the most commonly available vitamin B₆ supplement available. However, Vitamin B₆ related compounds include a variety of known analogues, metabolites, derivatives, or precursors, as described and discussed below.

Several studies have shown that P5P inhibits the effects of extra-cellular ATP in a number of different tissue types including the heart (Want et al, 1999, CanAm Bioresearch, 2003) vagus nerve (Trezise et al, 1994), vas deferens (Trezise et al, 1994) and smooth muscle cells (Lal et al, 1993). Previously published studies have shown that P5P inhibits ATP induced calcium influx in freshly isolated adult rat cardiomyocytes (Wang et al, 1999, CanAm Bioresearch, 2003), and the positive inotropic effects of ATP on isolated perfused rat hearts (Wang et al, 1999, CanAm Bioresearch, 2003). In addition, several published studies have shown that P5P inhibits ATP-binding to the cardiac sacrolemma (Wang et al, 1999), vagus nerve (Trezise et al, 1994) and vas deferens (Trezise et al, 1994).

There are currently seven known P2 purinergic receptors, P2X1, P2X2, P2X3, P2X4, P2X5, P2X6, and P2X7, which are known to form heteromeric complexes among themselves. ATP is known to act on P2X7. However, until now, it has not been known to what degree each P2-purinergic receptor is affected by P5P or any other vitamin B6 related compounds, and whether P2-purinergic receptors are affected by vitamin B6 related compounds in different manners. P5P has been shown to have some antagonist effect on P2 receptors, for example P2X7 receptor, but has been found to have significant species-dependent variability in its potency, which has raised questions of effectiveness and method of action (Hibell, 2001). The antagonist effect of P5P on P2X7 receptors is currently the basis of some uncertainty, as it is unclear whether it is a competitive, an irreversible, or a partially reversible antagonist (Michel, 2000).

High activity of P2X7 receptor has been found to be implicated in epithelial cancer (Coutinho-Silva et al, 2005), leukemia (Zhang et al, 2004), brain tumors (Guo et al, 2004), spinal cord injury (Wang, 2004), tuberculosis (Mancino et al, 2001), Alzheimer's Disease (Parvathenani et al, 2003), neurodegenerative diseases generally (LeFeuvre et al, 2002), autosomal recessive polycystic kidney disease (Hillman et al, 2004), diabetes, including type I diabetes (Elliott and Higgins, 2004), prostate cancer (Slater et al, 2004), osteoporosis, bone formation and resorption (Ke et al, 2003), rheumatoid arthritis, multiple sclerosis, myasthenia gravis, Crohn's disease, Septic shock, and periodontal infection (Mühl et al., 2003).

Interleukin-1 (IL-1) is an important inflammatory mediator produced in abundance by activated monocytes and macrophages (Dinarello, 1996). Inflammation and inflammation response is implicated in a large variety of diseases. An inflammation response, in turn, can trigger a wound healing response. However, chronic inflammation, or an inappropriate inflammatory response can lead to the formation of a chronic wound. Chronic inflammation may also lead to tissue damage through the excess release of reactive oxygen species. Inappropriate inflammatory response may also lead to abscess formation. Systemic inflammatory response syndrome, such as sepsis, occurs when inflammation overwhelms the whole organism. Inflammation of organs has been implicated in such diseases or disorders as appendicitis (inflammation of the appendix), arteritis (inflammation of the arteries), arthritis (inflammation of the joints), blepharitis (inflammation of the eyelids), bronchiolitis (inflammation of the bronchioles), bronchitis (inflammation of the bronchi), bursitis (inflammation of the bursa), cervicitis (inflammation of the cervix), cholangitis (inflammation of the bile duct), cholecystitis (inflammation of the gallbladder), chorioamnionitis (inflammation of the amniotic sac), colitis (inflammation of the colon), conjunctivitis (inflammation of the conjunctiva), cystitis (inflammation of the bladder), dacryoadenitis (inflammation of the lacrimal gland), dermatitis (inflammation of the skin), dermatomyositis (inflammation of the skin and muscles), encephalitis (inflammation of the brain), endocarditis (inflammation of the endocardium), endometritis (inflammation of the endometrium), enteritis (inflammation of the small intestine), enterocolitis (inflammation of the small and large intestines), epicondylitis (inflammation of the epicondyle), epididymitis (inflammation of the epididymis), fasciitis (inflammation of the fascia), fibrositis (inflammation of fibrous connective tissue), gastritis (inflammation of the stomach), gastroenteritis (inflammation of the stomach and small intestine), gingivitis (inflammation of the gingiva), hepatitis (inflammation of the liver), hidradenitis suppurativa (inflammation of the apocrine sweat glands), ileitis (inflammation of the ileum), iritis (inflammation of the iris), laryngitis (inflammation of the larynx), mastitis (inflammation of the mammary gland), meningitis (inflammation of the meninges), myelitis (inflammation of the spinal cord), myocarditis (inflammation of the myocardium), myositis (inflammation of the muscle), nephritis (inflammation of the kidney), omphalitis (inflammation of the umbilical cord), oophoritis (inflammation of the ovaries), orchitis (inflammation of the testicle), osteitis (inflammation of the bone), otitis (inflammation of the ear), pancreatitis (inflammation of the pancreas), parotitis (inflammation of the parotid gland), pericarditis (inflammation of the pericardium), peritonitis (inflammation of the peritoneum), pharyngitis (inflammation of the pharynx), pleuritis (inflammation of the pleura), phlebitis (inflammation of the veins), pneumonia/pneumonitis (inflammation of the lungs), proctitis (inflammation of the rectum), prostatitis (inflammation of the prostate), rhinitis (inflammation of the nasal lining), salpingitis (inflammation of the fallopian tubes), sinusitis (inflammation of the sinus of the skull), stomatitis (inflammation of the mouth), synovitis (inflammation of the synovial membrane), tendonitis (inflammation of the tendon), tonsillitis (inflammation of the tonsils), uveitis (inflammation of the uvea), vaginitis (inflammation of the vaginal mucosa), vasculitis (inflammation of the blood vessels or lymph vessels), and vulvitis (inflammation of the vulva).

IL-1 biological activity is derived from two related but distinct polypeptides, IL-1α and IL-1β (Dinarello, 1996 and 1998). IL-1β activity has been correlated to ATP levels, as discussed below. Interestingly, lipopolysaccharide/ATP-induced secretion of IL-1β ex vivo has been found to be completely suppressed in blood cultures obtained from P2X7 knockout mice, however, the relationship between P2X7 and IL-1β is poorly understood (Muhl, 2003).

Human IL-1 is synthesized as a 31 kDa pro-cytokine that is incompetent to bind to the type 1 IL-1 receptor (Mosely et al, 1987). To gain activity, pro-IL-1β must be cleaved by caspase-1 to yield a 17 kDa carboxyl terminus-derived polypeptide (Thornberry et al, 1992; Ceretti et al, 1992). IL-1β is released from monocytes and macrophages via an atypical secretory mechanism that does not involve the endoplasmic reticulum and Golgi complex (Rubartelli et al, 1990). Release of IL-1β from cells stimulated to produce this cytokine is generally an inefficient process. The majority of newly synthesized cytokine molecules remains cell associated and/or are degraded (Hogquist et al, 1991; Perregaux et al, 1998; Chin et al, 1993). To promote the efficient proteolytic cleavage of pro-IL-1 and release of the 17 kDa mature peptide, the cytokine-producing cells must be treated with a secretion stimulus such as adenosine triphosphate (ATP; Perregaux et al, 1998; Laliberte et al, 1999; Grahames et al, 1999).

Extracellular ATP has been found to markedly accelerate the rate of processing and release of IL-1β in both monocytes and macrophages that have been primed with lipopolysacchamides (LPS; Laliberte et al, 1999; Grahames et al, 1999; Perregaux et al, 1998). The ATP induced changes are mediated via the activation of P2X7 purinergic receptors (Grahames et al, 1999; Labassi et al, 2002), which, in turn, accelerate the processing and release of IL-1β (Perregaux et al, 1992; Perregaux et al, 1994; Perregaux et al, 1998).

### SUMMARY OF THE INVENTION

The present invention is directed to a novel use for pyridoxal-5-phosphate (P5P).

More particularly, the present invention is directed to a method of modulating P2X7 comprising administering a therapeutically effective amount of P5P or a pharmaceutically acceptable salt thereof to a patient in need thereof.

Another aspect of the present invention is the method wherein the therapeutically effective amount of pyridoxal-5′-phosphate is between 0.5 and 50 mg/kg body weight.

Another aspect of the present invention is the method wherein the therapeutically effective amount of pyridoxal-5′-phosphate is between 1 and 15 mg/kg body weight.

Another aspect of the present invention is the method wherein the patient is human.

Another aspect of the present invention is the method wherein the patient has a disease or metabolic disorder.

Another aspect of the present invention is the method wherein the metabolic disorder is epithelial cancer, leukemia, brain tumor, spinal cord injury, tuberculosis, Alzheimer's Disease, neurodegenerative disease, autosomal recessive polycystic kidney disease, diabetes, prostate cancer, osteoporosis, autoimmune disease, rheumatoid arthritis, multiple sclerosis, myasthenia gravis, Crohn's disease, Septic shock, or periodontal infection.

Another aspect of the present invention is the method wherein the autoimmune disease is selected from lupus erythematosis and rheumatoid arthritis.

Another aspect of the present invention is the method wherein the diabetes is type I diabetes.

Another aspect of the present invention is directed to a method of controlling or mediating inflammation response comprising administering a therapeutically effective amount of P5P or a pharmaceutically acceptable salt thereof to a patient in need thereof.

Another aspect of the present invention is directed to a method of decreasing or mediating IL-1β levels in a patient in need thereof comprising administering a therapeutically effective amount of P5P or a pharmaceutically acceptable salt thereof to the patient.

Another aspect of the present invention is the method wherein the disease or metabolic disorder is an inflammatory disease or disorder.

Another aspect of the present invention is the method wherein the disease or metabolic disorder is a disease or disorder characterized by having IL-1β levels that are higher than normal, either locally or systemically.

Another aspect of the present invention is the method wherein the inflammatory disease or disorder is a chronic wound, chronic inflammation, abscess formation, systemic inflammatory response syndrome, including sepsis, appendicitis, arteritis, arthritis, blepharitis, bronchiolitis, bronchitis, bursitis, cervicitis, cholangitis, cholecystitis, chorioamnionitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, gingivitis, hepatitis, hidradenitis suppurativa, ileitis, iritis, laryngitis, mastitis, meningitis, myelitis, myocarditis, myositis, nephritis, omphalitis, oophoritis, orchitis, osteitis, otitis, pancreatitis, parotitis, pericarditis, peritonitis, pharyngitis, pleuritis, phlebitis, pneumonia/pneumonitis, proctitis, prostatitis, rhinitis, salpingitis, sinusitis, stomatitis, synovitis, tendonitis, tonsillitis, uveitis, vaginitis, vasculitis, or vulvitis.

Another aspect of the present invention is the method wherein the inflammatory disease or disorder is one specifically characterized by increased levels of IL-1β. Such diseases or disorders include inflammatory bowel disease, ulcerative colitis, Crohn's disease, Sjogren's Syndrome, bone erosion, neuroinflammatory diseases such as Alzheimer's disease, Parkinson's disease, and traumatic brain injury, as well as periodontal disease.

Another aspect of the present invention is the use of a therapeutically effective amount of P5P or a pharmaceutically acceptable salt thereof for the treatment or prevention of a metabolic disorder selected from the group consisting of: epithelial cancer, leukemia, brain tumor, spinal cord injury, tuberculosis, Alzheimer's Disease, neurodegenerative disease, autosomal recessive polycystic kidney disease, diabetes, prostate cancer, osteoporosis, autoimmune disease, rheumatoid arthritis, multiple sclerosis, myasthenia gravis, Crohn's disease, Septic shock, and periodontal infection.

Another aspect of the present invention is the use of a therapeutically effective amount of P5P or a pharmaceutically acceptable salt thereof for the treatment or prevention of an inflammatory disease or disorder selected from the group consisting of: chronic wound, chronic inflammation, abscess formation, systemic inflammatory response syndrome, including sepsis, appendicitis, arteritis, arthritis, blepharitis, bronchiolitis, bronchitis, bursitis, cervicitis, cholangitis, cholecystitis, chorioamnionitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, gingivitis, hepatitis, hidradenitis suppurativa, ileitis, iritis, laryngitis, mastitis, meningitis, myelitis, myocarditis, myositis, nephritis, omphalitis, oophoritis, orchitis, osteitis, otitis, pancreatitis, parotitis, pericarditis, peritonitis, pharyngitis, pleuritis, phlebitis, pneumonia/pneumonitis, proctitis, prostatitis, rhinitis, salpingitis, sinusitis, stomatitis, synovitis, tendonitis, tonsillitis, uveitis, vaginitis, vasculitis, or vulvitis.

Another aspect of the present invention is the use of a therapeutically effective amount of P5P or a pharmaceutically acceptable salt thereof for the treatment or prevention of an inflammatory disease characterized by higher than normal levels of IL-1β selected from the group consisting of inflammatory bowel disease, ulcerative colitis, Crohn's disease, Sjogren's Syndrome, bone erosion, neuroinflammatory diseases (such as Alzheimer's disease, Parkinson's disease, and traumatic brain injury), and periodontal disease.

Another aspect of the present invention is the use wherein the therapeutically effective amount of pyridoxal-5′-phosphate is between 0.5 and 50 mg/kg body weight.

Another aspect of the present invention is the use wherein the therapeutically effective amount of pyridoxal-5′-phosphate is between 1 and 15 mg/kg body weight.

Another aspect of the present invention is a kit comprising P5P and instructions for the use of the same for treatment of a disease or disorder described above.

Another aspect of the present invention is the use of P5P in the preparation of a medicament for the treatment of any of the diseases or disorders described above.

### BRIEF DESCRIPTION OF THE DRAWINGS

FIG. 1 shows cell viability in parent HEK 293 cells treated with varying concentrations of ATP.

FIG. 2 shows cell viability in HEK 293 cells stably expressing rat P2X7, treated with varying concentrations of ATP.

FIG. 3 shows cell viability in parent HEK 293 cells, treated with 2 μM of ATP 30 minutes following treatment with varying concentrations of P5P.

FIG. 4 shows cell viability in HEK 293 cells stably expressing rat P2X7, treated with 0.4 μM of ATP 30 minutes following treatment with varying concentrations of P5P.

FIG. 5 shows the effect of various concentrations of pyridoxine (0, 0.5, 1.5, 5, 15, 50, 150 μM) on ATP (5 mM) induced IL-1β processing and release from PMA/LPS stimulated THP-1 cells.

FIG. 6 shows the effect of various concentrations of pyridoxine phosphate (0, 0.5, 1.5, 5, 15, 50, 150 μM) on ATP (5 mM) induced IL-1β processing and release from PMA/LPS stimulated THP-1 cells.

FIG. 7 shows the effect of various concentrations of pyridoxal (0, 0.5, 1.5, 5, 15, 50, 150 μM) on ATP (5 mM) induced IL-1β processing and release from PMA/LPS stimulated THP-1 cells.

FIG. 8 shows the effect of various concentrations of pyridoxal phosphate (0, 0.5, 1.5, 5, 15, 50, 150 μM) on ATP (5 mM) induced IL-1β processing and release from PMA/LPS stimulated THP-1 cells.

FIG. 9 shows the effect of various concentrations of pyridoxamine (0, 0.5, 1.5, 5, 15, 50, 150 μM) on ATP (5 mM) induced IL-1β processing and release from PMA/LPS stimulated THP-1 cells.

FIG. 10 shows the effect of various concentrations of pyridoxamine phosphate (0, 0.5, 1.5, 5, 15, 50, 150 μM) on ATP (5 mM) induced IL-1β processing and release from PMA/LPS stimulated THP-1 cells.

### DESCRIPTION OF THE INVENTION

The present inventor has shown that P5P functions as a P2-purinergic receptor antagonist. Furthermore, the present inventor has now shown that P5P functions as an efficient P2X7 receptor antagonist.

The present inventor has also found that P5P functions as an inhibitor of IL-1β release or expression. Surprisingly, the present inventor has also found that this IL-1β inhibition activity is not a characteristic of other Vitamin B6 related compounds, such as pyridoxal, pyridoxamine, and the like, and appears to be unique to P5P.

As used herein, “vitamin B6 related compound”, means any vitamin B6 precursor, metabolite, derivative, or analogue thereof. Examples of vitamin B6 related compounds include but are not limited to pyridoxal-5-phosphate (P5P), pyridoxal, pyridoxine, pyridoxine phosphate, pyridoxamine, and pyridoxamine phosphate.

The invention also includes pharmaceutically acceptable salts of the compounds of the invention. The compounds of the invention are capable of forming both pharmaceutically acceptable acid addition and/or base salts. Pharmaceutically acceptable acid addition salts of the compounds of the invention include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and di-carboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate, n-methyl glucamine, etc. (see Berge et al, 1977). The term “pharmaceutically acceptable salts” also includes any pharmaceutically acceptable base salt including, but not limited to, amine salts, trialkyl amine salts and the like. Such salts can be formed quite readily by those skilled in the art using standard techniques.

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention. Base salts are formed with metals or amines, such as alkali and alkaline earth metals or organic amines. Examples of metals used as cations include, but are not limited to, sodium, potassium, magnesium, and calcium. Examples of suitable amines are N,N′-dibenzylethylenediamine, chloroprocaine, choline, diethanolamine, ethylenediamine, N-methylglucamine, and procaine.

Some of the compounds described herein contain one or more asymmetric centers and this may give raise to enantiomers, disasteriomers, and other stereroisomeric forms which may be defined in terms of absolute stereochemistry as (R)- or (S)-. The present invention is meant to include all such possible diasteriomers and enantiomers as well as their racemic and optically pure forms. Optically active (R)- and (S)-isomers may be prepared using chiral synthons or chiral reagents, or resolved using conventional techniques. When the compounds described herein contain olefinic double bonds or other centres of geometric symmetry, and unless specified otherwise, it is intended that the compounds include both E and A geometric isomers. Likewise all tautomeric forms are intended to be included.

Although it is possible for a compound suitable for use in methods of the invention to be administered alone in a unit dosage form, preferably the compound is administered in admixture as a pharmaceutical composition suitable for use in methods of the invention. A pharmaceutical composition comprises a pharmaceutically acceptable carrier and a compound. A pharmaceutically acceptable carrier includes, but is not limited to, physiological saline, ringers, phosphate buffered saline, and other carriers known in the art. Pharmaceutical compositions may also include additives, for example, stabilizers, antioxidants, colorants, excipients, binders, thickeners, dispersing agents, readsorpotion enhancers, buffers, surfactants, preservatives, emulsifiers, isotonizing agents, and diluents. Pharmaceutically acceptable carriers and additives are chosen such that side effects from the pharmaceutical compound are minimized and the performance of the compound is not canceled or inhibited to such an extent that treatment is ineffective.

Methods of preparing pharmaceutical compositions containing a pharmaceutically acceptable carrier and a compound suitable for use in methods of the invention are known to those of skill in the art. All methods may include the step of bringing the compound in association with the carrier and additives. In general, the formulations are prepared by uniformly and intimately bringing the compound of the invention into association with a liquid carrier or a finely divided solid carrier or both, and then, if necessary, shaping the product into the desired unit dosage form.

A physician or veterinarian of ordinary skill can readily determine a subject who is or may be suffering from a disease state that could implicate P2X7 dependent pathways such as P2X7 dependent apoptosis, inflammation response, or increased extracellular levels of IL-1β. Regardless of the route of administration selected, P5P or a pharmaceutically acceptable salt thereof can be formulated into pharmaceutically acceptable unit dosage forms by conventional methods known to the pharmaceutical art. An effective but nontoxic quantity of the compound can be employed in treatment.

The therapeutic compound of P5P or a pharmaceutically acceptable salt thereof can be administered in enteral unit dosage forms, such as, for example, tablets, sustained-release tablets, enteric coated tablets, capsules, sustained-release capsules, enteric coated capsules, pills, powders, granules, solutions, and the like. They can also be administered parenterally, such as, for example, subcutaneously, intramuscularly, intradermally, intramammarally, intravenously, and other administrative methods known in the art.

Although it is possible for P5P or a pharmaceutically acceptable salt thereof as described above to be administered alone in a unit dosage form, preferably the compound is administered in admixture as a pharmaceutical composition.

By an “effective” amount or a “therapeutically effective amount” of a drug or pharmacologically active agent is meant a nontoxic but sufficient amount of the drug or agent to provide the desired effect. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

A therapeutic compound can be administered, for example, after a disease state has been diagnosed. A therapeutic compound can also be administered before the onset of an event or disease state.

A therapeutic compound as defined above can be formulated into a pharmaceutical composition for use in methods of the invention.

The ordinarily skilled physician or veterinarian will readily determine and prescribe a therapeutically effective amount of P5P or a pharmaceutically acceptable salt thereof to modulate P2X7 dependent pathways, inflammation response, or extracellular levels of IL-1β. In so proceeding, the physician or veterinarian could employ relatively low dosages at first, subsequently increasing the dose until a maximum response is obtained. Typically, the particular disease, the severity of the disease, the extent of cell death or stress, the compound to be administered, the route of administration, and the characteristics of the mammal to be treated, for example, age, sex, and weight, can be considered in determining the effective amount to administer. In one embodiment of the invention, a therapeutic amount is in a range of about 0.1-100 mg/kg of a patient's body weight, in another embodiment in the range of about 0.5-50 mg/kg of a patient's body weight, per daily dose. The compound can be administered for periods of short or long duration. Although some individual situations can warrant to the contrary, short-term administration, for example, 30 days or less, of doses larger than 25 mg/kg of a patient's body weight is chosen when compared to long-term administration. When long-term administration, for example, months or years, is utilized, the suggested dose generally should not exceed 25 mg/kg of a patient's body weight.

A therapeutically effective amount of P5P or a pharmaceutically acceptable salt thereof for modulating P2X7 activity, inflammation response, or extracellular levels of IL-1β, or for treating a disease, disorder or symptom in which P2X7, an inflammation response, or IL-1β is implicated, can be administered prior to, concurrently with, or after the onset of the disease, disorder, or symptom.

A therapeutic compound of the invention can be administered concurrently with or subsequent to compounds that are already known to be suitable for treating the disease state. “Concurrent administration” and “concurrently administering” as used herein includes administering a therapeutic compound and a known therapy in admixture such as, for example, in a pharmaceutical composition or in solution, or as separate components, such as, for example, separate pharmaceutical compositions or solutions administered consecutively, simultaneously, or at different times but not so distant in time such that the therapeutic compound and the known therapy cannot interact and a lower dosage amount of the active ingredient cannot be administered.

It is to be understood that this invention is not limited to specific dosage forms, carriers, or the like, and as such may vary. It is also to be understood that the terminology used herein is for the purpose of describing particular embodiments only, and is not intended to be limiting.

P5P or a pharmaceutically acceptable salt thereof can be administered orally. Preferred oral dosage forms contain a therapeutically effective unit dose of each active agent, wherein the unit dose is suitable for a once-daily oral administration. The therapeutic effective unit dose of any of the active agents will depend on number of factors, which will be apparent to those skilled in the art and in light of the disclosure herein. In particular these factors include: the identity of the compound to be administered, the formulation, the route of administration employed, the patient's gender, age, and weight, and the severity of the condition being treated and the presence of concurrent illness affecting the gastrointestinal tract, the hepatobillary system and the renal system. Methods for determining dosage and toxicity are well known in the art with studies generally beginning in animals and then in humans if no significant animal toxicity is observed. The appropriateness of the dosage can be assessed by monitoring triglyceride levels. Where the dose provided does not cause triglyceride levels to decline to normal or tolerable levels, following at least 2 to 4 weeks of treatment, the dose can be increased.

Methods of preparing pharmaceutical compositions containing a pharmaceutically acceptable carrier and P5P or a pharmaceutically acceptable salt thereof are known to those of skill in the art.

All methods can include the step of bringing the compound of the invention in association with the carrier and additives. The formulations generally are prepared by uniformly and intimately bringing the compound of the invention into association with a liquid carrier or a finely divided solid carrier or both, and then, if necessary, shaping the product into the desired unit dosage form.

Generally, a solution of a therapeutic compound, for example P5P, may be prepared by simply mixing P5P with a pharmaceutically acceptable solution, for example, buffered aqueous saline solution at a neutral or alkaline pH, at a temperature of at least room temperature and under sterile conditions. In one embodiment, the P5P solution is prepared immediately prior to administration to the mammal. However, if the P5P solution is prepared at a time more than immediately prior to the administration to the mammal, the prepared solution can be stored under sterile, refrigerated conditions. Furthermore, the P5P solution can be stored in containers suitable for protecting the P5P solution from the light, such as amber-colored vials or bottles.

The compounds of the invention may be particularly useful in animal disorders (veterinarian indications), and particularly mammals.

The invention further provides diagnostic and pharmaceutical packs and kits comprising one or more containers filled with one or more of the ingredients of the aforementioned compositions of the invention. Associated with such container(s) can be a notice in the form prescribed by a governmental agency regulating the manufacture, use or sale of pharmaceuticals or biological products, reflecting approval by the agency of the manufacture, use or sale of the product for human administration.

As previously discussed, high activity of P2X7 receptor has been found to be implicated in a wide variety of diseases, such as epithelial cancer, leukemia, brain tumors, spinal cord injury, tuberculosis, Alzheimer's Disease, neurodegenerative diseases generally, autosomal recessive polycystic kidney disease, diabetes, including type I diabetes, prostate cancer, osteoporosis, bone formation and resorption, rheumatoid arthritis, multiple sclerosis, myasthenia gravis, Crohn's disease, Septic shock, and periodontal infection. This suggested to the Inventors that P2X7 receptor may be involved in these disease states.

The inventors have surmised that therefore inhibition of ATP-mediated P2X7 dependent pathways such as apoptosis may be desirable in the prevention, maintenance, treatment or cure of these disease states. Thus, inhibitors of ATP-mediated P2X7 receptor-dependent pathways such as apoptosis would be desirable, as useful therapeutic agents for the prevention, maintenance, treatment or cure of diseases in which P2X7-dependent pathways such as apoptosis are a factor, for example, epithelial cancer, leukemia, brain tumors spinal cord injury, tuberculosis, Alzheimer's Disease, neurodegenerative diseases, autosomal recessive polycystic kidney disease, diabetes, including type I diabetes, prostate cancer, osteoporosis, bone formation and resorption, rheumatoid arthritis, multiple sclerosis, myasthenia gravis, Crohn's disease, Septic shock, and periodontal infection.

Surprisingly, the present inventors have, in a series of elegant experiments, discovered that P5P protects cells from ATP-mediated, P2X7 dependent apoptosis, an indicator of the shutting down of ATP-mediated, P2X7 dependent pathways. Thus P5P can be used to inhibit the P2X7-dependent pathways, such as apoptosis, and can prevent the triggering of such pathways in diseases in which P2X7 has been implicated. P5P can therefore be used as effective treatments for the modulation of P2X7, and for diseases in which prevention of P2X7-dependent pathways such as apoptosis is desirable, such as epithelial cancer, leukemia, brain tumors spinal cord injury, tuberculosis, Alzheimer's Disease, neurodegenerative diseases, autosomal recessive polycystic kidney disease, diabetes, including type I diabetes, prostate cancer, osteoporosis, bone formation and resorption, rheumatoid arthritis, multiple sclerosis, myasthenia gravis, Crohn's disease, Septic shock, and periodontal infection.

The inventors have also surmised that inhibition of ATP-mediated P2X7 pathways can result in a mediation of inflammation response, and a modulation of extracellular levels of IL-1β. Thus, inhibitors of ATP-mediated P2X7 receptor-dependent pathways such as apoptosis would be desirable, as useful therapeutic agents for the prevention, maintenance, treatment or cure of diseases or disorders in which it is desired to reduce an inflammatory response, diseases in which IL-1β is implicated, or diseases in which a reduction in levels of extracellular IL-1β is desired, in order to prevent, maintain, treat or cure the disease. Such diseases or disorders include classic inflammatory diseases such as chronic wound, chronic inflammation, abscess formation, systemic inflammatory response syndrome, including sepsis, appendicitis, arteritis, arthritis, blepharitis, bronchiolitis, bronchitis, bursitis, cervicitis, cholangitis, cholecystitis, chorioamnionitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, gingivitis, hepatitis, hidradenitis suppurativa, ileitis, iritis, laryngitis, mastitis, meningitis, myelitis, myocarditis, myositis, nephritis, omphalitis, oophoritis, orchitis, osteitis, otitis, pancreatitis, parotitis, pericarditis, peritonitis, pharyngitis, pleuritis, phlebitis, pneumonia/pneumonitis, proctitis, prostatitis, rhinitis, salpingitis, sinusitis, stomatitis, synovitis, tendonitis, tonsillitis, uveitis, vaginitis, vasculitis, or vulvitis. Such diseases or disorders also include diseases and disorders in which high extracellular IL-1β levels have been specifically implicated, such as inflammatory bowel disease, ulcerative colitis, Crohn's disease, Sjogren's Syndrome, bone erosion, neuroinflammatory diseases such as Alzheimer's disease, Parkinson's disease, and traumatic brain injury, as well as periodontal disease.

Surprisingly, the present inventors have, in a series of elegant experiments, discovered that P5P decreases processing and release of IL-1β. Thus P5P can be used to inhibit the P2X7-dependent release of IL-1β, and the resultant inflammatory cascade. P5P can therefore be used as effective treatments for the modulation of IL-1β, and the modulation of inflammation in general, and for diseases or disorders in which modulation of IL-1β or modulation of inflammation is desirable, such as chronic wound, chronic inflammation, abscess formation, systemic inflammatory response syndrome, including sepsis, appendicitis, arteritis, arthritis, blepharitis, bronchiolitis, bronchitis, bursitis, cervicitis, cholangitis, cholecystitis, chorioamnionitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, gingivitis, hepatitis, hidradenitis suppurativa, ileitis, iritis, laryngitis, mastitis, meningitis, myelitis, myocarditis, myositis, nephritis, omphalitis, oophoritis, orchitis, osteitis, otitis, pancreatitis, parotitis, pericarditis, peritonitis, pharyngitis, pleuritis, phlebitis, pneumonia/pneumonitis, proctitis, prostatitis, rhinitis, salpingitis, sinusitis, stomatitis, synovitis, tendonitis, tonsillitis, uveitis, vaginitis, vasculitis, vulvitis, and diseases and disorders in which IL-1β has specifically been implicated, such as inflammatory bowel disease, ulcerative colitis, Crohn's disease, Sjogren's Syndrome, bone erosion, neuroinflammatory diseases such as Alzheimer's disease, Parkinson's disease, and traumatic brain injury, as well as periodontal disease.

Surprisingly and unexpectedly, the present inventors have found that most tested B6 related compounds, such as pyridoxine, pyridoxine phosphate, pyridoxal, pyridoxamine, and pyridoxamine phosphate were not effective in inhibiting IL-1β processing and release. A surprising discovery is that P5P is uniquely effective, among the B6 related compounds tested, in inhibiting the processing and release of IL-1β.

This invention will be further characterized by the following examples. These examples are not meant to limit the scope of the invention, which has been fully set forth in the foregoing description. Variations within the scope of the invention will be apparent to those skilled in the art.

### Example 1

### Induction of Cell Death in HEK 293 P2X7 (Rat) Cells with ATP

HEK 293 cells were stably transfected to express rat P2X7 receptor, using standard tissue culture techniques (Surprenant et al, 1996). The cell line was known to not express any of the other P2X receptors (Schachter et al, 1997). Stable expression of P2X7 was confirmed by Western blot (results not shown).

Parent (HEK 293) cells, and HEK 293 cells expressing rat P2X7 (HEK 293 P2X7 (rat) cells) were treated with ATP (Sigma, A9062) to determine the effects of ATP on the viability of the cells. Cell viability was determined using an MTT assay (Wen, 2003). Cells were treated with between 0 and 2 μM of ATP. MTT was added to the cell cultures 180 minutes after ATP treatment. The MTT assay was performed 60 minutes after the addition of MTT. Cell viability was directly correlatable to the absorbance of 550 nm light by the cell culture, with a higher absorbance meaning higher cell viability. The cell culture media was removed and the cells were solubilized using DMSO. The absorbance of the solubilized cells was read.

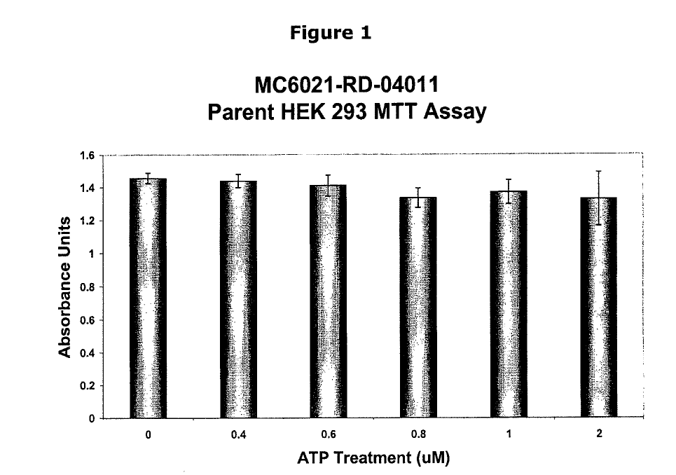

FIG. 1 shows the cell viability in parent HEK 293 cells. Up to 2 μM of ATP treatment did not significantly affect the viability of parent cells.

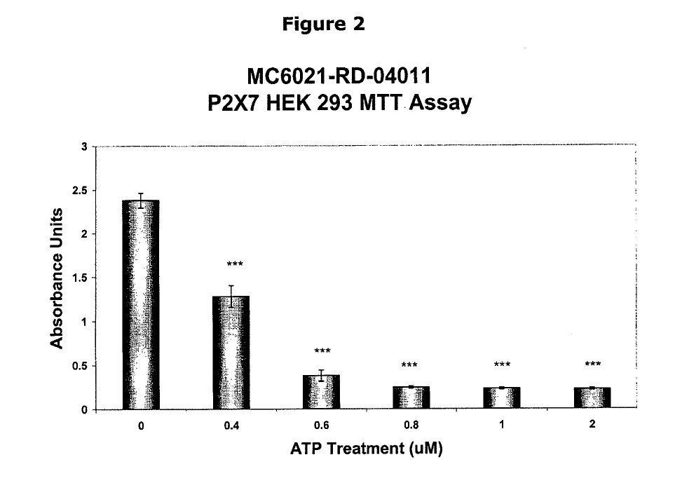

FIG. 2 shows the cell viability in HEK 293 P2X7 (rat) cells. Cells expressing rat P2X7 exhibited significant loss in cell viability starting at 0.4 μM of ATP treatment.

This Example establishes the conditions required to trigger P2X7-dependent pathways, such as apoptosis, using, as an indicator, the killing of cells in an ATP-mediated, P2X7-dependent cell death.

### Example 2

### P5P Treatment Protects Cells from ATP-Mediated, P2X7-Dependent Cell Death

HEK 293 and HEK 293 P2X7 (rat) cells were treated with 0 to 50 μM of P5P, a vitamin B6 related compound, in sodium salt form (Chemistry Department, CanAm Bioresearch Inc.). Thirty minutes later, cells were treated with 0.4 μM ATP, to determine the effects of ATP on the viability of the MC-1 treated cells. Control cells received sham treatment of P5P, and sham treatment of ATP.

Cell viability was determined using an MTT assay [16]. MTT was added to the cell cultures 180 minutes after ATP treatment. The MTT assay was performed 60 minutes after the addition of MTT. Cell viability was directly correlatable to the absorbance of 550 nm light by the cell culture, with a higher absorbance meaning higher cell viability.

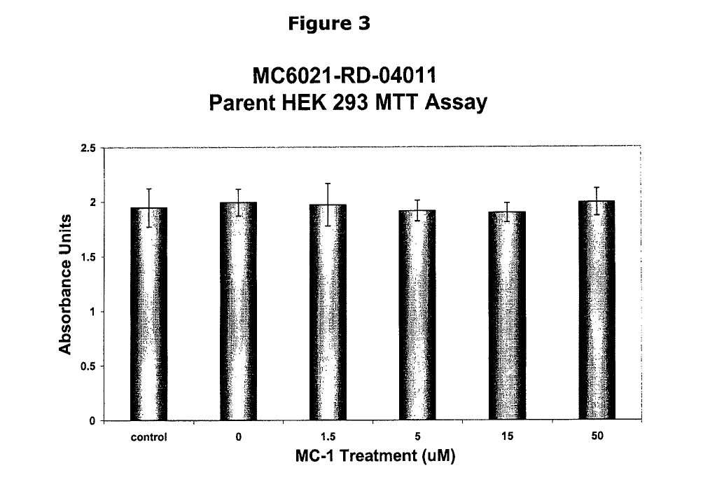

FIG. 3 shows the cell viability in HEK 293 cells. Control cells received sham treatment of P5P and sham treatment of ATP. Cells in the “0” column received sham treatment of P5P but were treated with ATP. As seen in FIG. 1, 0.4 μM of ATP treatment did not significantly affect the viability of HEK 293 cells. As such, P5P treatment also did not have any significant effect on the viability of the cells.

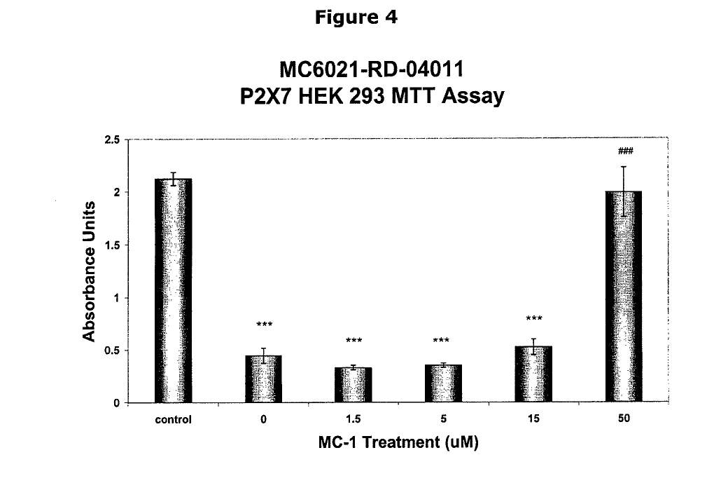

FIG. 4 shows the cell viability in HEK 293 P2X7 (rat) cells. As consistent with FIG. 2, cells expressing rat P2X exhibited significant loss in cell viability when treated with ATP. Treatment of from 1.5 to 15 μM of P5P did not significantly affect this viability. However, when treated with 50 μM of P5P, half an hour before the ATP treatment, cell viability was significantly improved. In fact, there was no significant difference between the viability of ATP treated cells that had been “innoculated” with P5P, and control cells.

This Example establishes that P5P treatment at sufficient concentration protects cells from ATP-mediated, P2X7 dependent, pathways such as apoptosis.

### Example 3

### P5P Inhibits ATP-Induced Processing and Release of IL-1β from PMA/LPS Stimulated THP-1 Cells

THP-1 non-adherent, monocytic cells (ATCC) were pre-treated with phorbol-12-myristate-13-acetate (PMA), which differentiated the cells into macrophage-like cells. Briefly, approximately 8×10⁶ THP-1 cells were harvested, pelleted, resuspended in media to which PMA was added at a final concentration of 0.5 μM. The cells were incubated for 3 hours, then collected, washed, and plated.

The next day, the cells were treated with lipopolysaccharide (LPS) to stimulate IL-1β production. Briefly, the media was removed and replaced with RPI media+10% fetal calf serum containing 100 ng/ml of LPS. Cells were incubated for an additional 24 hours.

Media was then changed, and the test compound (pyridoxine phosphate, pyridoxal, P5P, pyridoxamine, or pyridoxamine phosphate) was added to a final concentration of 0, 0.5, 1.5, 5, 15, 50 or 150 μM. Cells were pretreated for 30 minutes, then the media was replaced with media containing 5 mM ATP as well as the test compound. Cells were exposed to ATP for one hour. Media was then collected and the amount of IL-1β in the media was determined using a human IL-1β ELISA (R&D Systems).

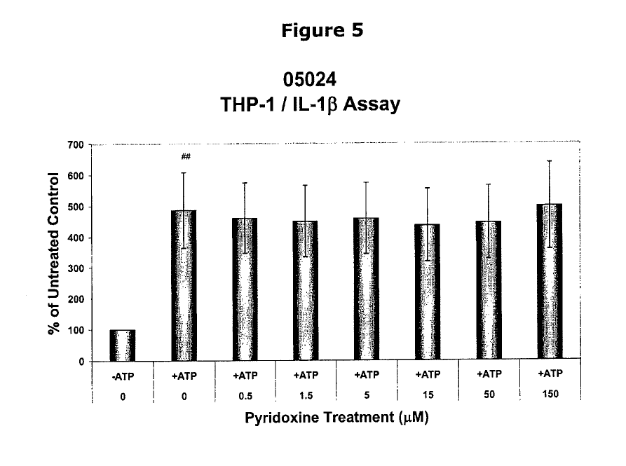

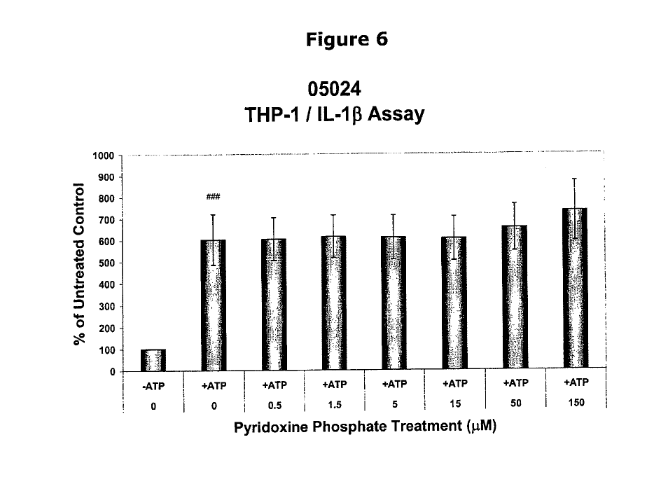

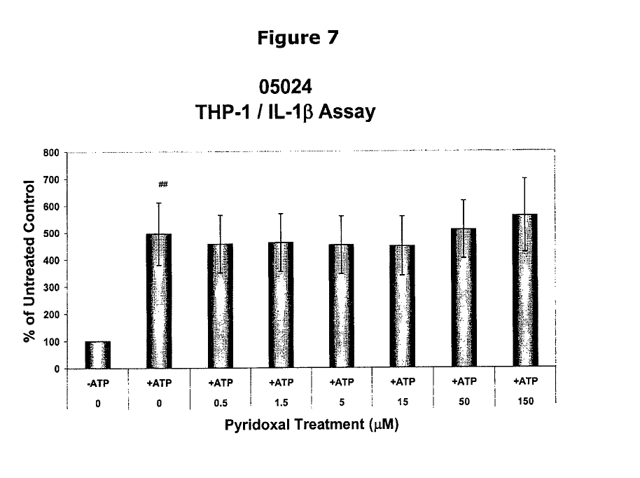

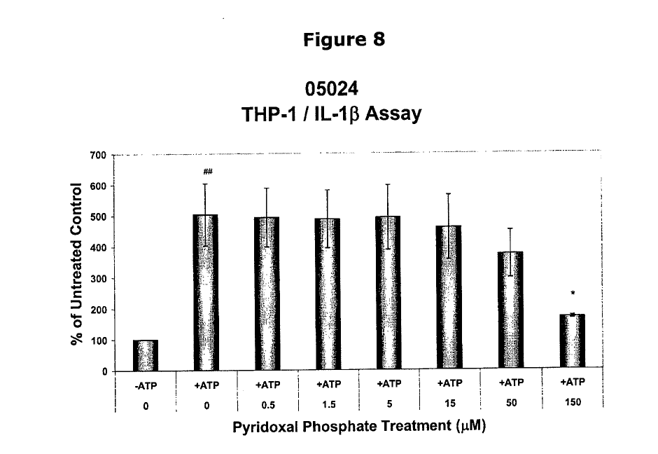

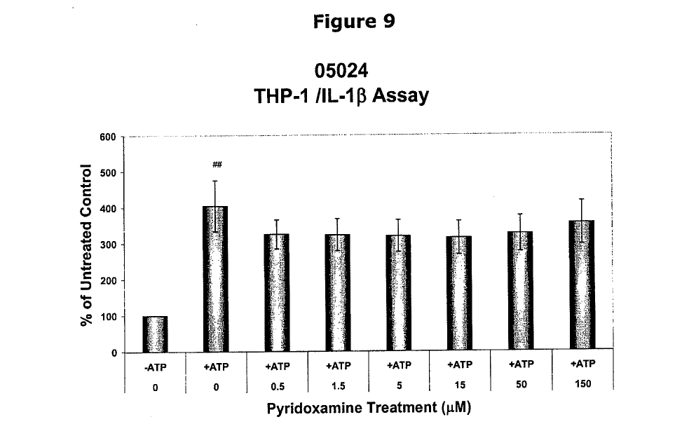

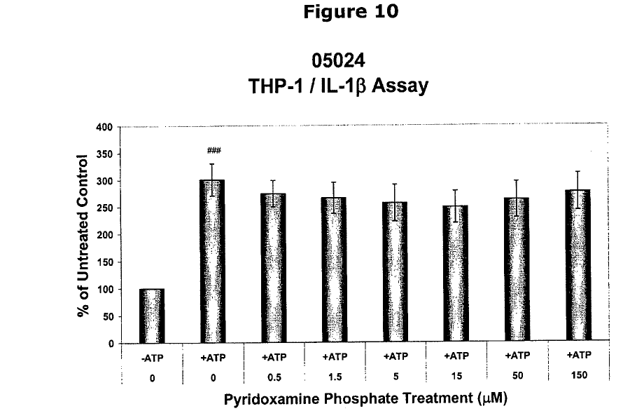

Results were expressed as the mean±sem. Statistical analysis was performed using bulk t-test (Medistats). FIGS. 5-10 show the effect of the test compound (pyridoxine, pyridoxine phosphate, pyridoxal, P5P, pyridoxamine, and pyridoxamine phosphate, respectively) at various concentrations (0, 0.5, 1.5, 5, 15, 50 and 150 μM on ATP (5 mM) induced IL-1β processing and release from PMA/LPS stimulated THP-1 cells. IL-1β levels were measured in the cell culture media by ELISA and the results are expressed as a percent of the untreated (baseline) controls.

The results showed that treatment of PMA/LPS primed THP-1 cells with ATP caused a significant increase in the processing and release of IL-1β into the culture media (#=P<0.05, ##=P<0.01 and ###=P<0.001). Treatment of the cells with pyridoxine, pyridoxine phosphate, pyridoxal, pyridoxamine, or pyridoxamine phosphate had no significant effect on the ATP-induced processing and release of IL-1β at any of the concentrations tested (*=P<0.05, **=P<0.01 and ***=P<0.001). Treatment of the cells with P5P, however, significantly inhibited the ATP induced processing and release of IL-1β (*=p<0.05,**=P<0.01 and ***=P<0.001).

### Example 4

### P5P Treatment Protects Cells from BzATP-Mediated, P2X7-Dependent Cell Death

Examples 1-2 are repeated, substituting BzATP for ATP. BzATP is known to be a specific agonist of P2X7 receptors. This Example yields results consistent with Examples 1-4, further establishing that the pathway utilized is a P2X7 dependent pathway.

### Example 5

### Effect of P5P on the Cytokine Response of Lipopolysaccharide Treated Rats

Sprague-Dawley rats weighing approximately 300 g are treated with 1, 3, or 10 mg/kg P5P (or a saline control). Thirty minutes later (t=30), the animals are given a 50 μg/kg intraperitoneal injection of LPS (or a saline control). Temperature of the animals is measured, and a blood sample is collected at 240 minutes post-treatment (t=240).

Blood samples are transferred to heparinized tubes and plasma is separated by centrifugation. Plasma is assayed for IL-1β, [IL-6 and TNF-α].

The experiment confirms that LPS treatment in vivo causes a significant increase in the processing and release of IL-1β [IL-6 and TNF-α] into the plasma. In vivo treatment with P5P significantly inhibits processing and release of IL-1β in vivo.

### REFERENCES

Apasov et al (1995) Immunol Rev 146:5-19.
Berge et al., (1977) J Pharmaceutical Science 66:1-19
CanAm Bioresearch (2003) MC1-PCe-02-03-01-47.
CanAm Bioresearch (2003) MC1-PCe-08-03-01-360.
Ceretti D P et al (1992) Science 256:97-100.
Chessell I P et al (1998) Br J Pharmacol 124:1314-1320.
Chin J and Kostura M J (1993) J Immunol 151:5574-5585.
Coutinho-Silva R. et al. (2005) Blood 87: 2095-2147.
Dinarello C A (1998) Int Rev Immunol 16:457-499.
Di Virigilio et al (2001) Blood 97:587-600.
Dubyak G R et al (1993) Am I Physiol 265:577-606.
Elliott J I and Higgins C F (2004) Diabetes 53(8):2012-7
Grahames C B A et al (1999) Br J Pharm 127:1915-1921.
Gudipaty L et al (2003) Am J Physiol Cell Physiol 285:286-299.
Guo L H et al (2004) J. Neuroimmunol 152(1-2):67-72
Harden T K et al (1994) Annu Rev Pharmacol Toxicol 35:541-579.
Hibell et al., (2001) J. Pharmacol. Exp. Therap. 296:947-957
Hillman K A et al (2004) Biochem Biophys Res Commun 322(2):434-9
Hogquist K A et al (1991) J Immunol 147:2181-2186.
Humphreys B D et al (1998) Mol Pharmacol 54:22-32.
Ke H Z et al 2003 Mol Endocrinol 17(7) 1356-67
Labasi J M et al (2002) J Immunol 168:6436-6445.
Lal K J et al (1993) Clin Exper Hypertension 15:489-500.
Laliberte R E, et al (1999) J Biol Chem 274:36944-36951.
LeFeuvre R et al (2002) Eur J Pharmacol 447(2-3) 261-9
Mancino G et al (2001) J Biol Regul Homeost Agents 15(3) 286-93
Michel A. D. et al (2000) British J. Pharmacol. 130, 513-520
Mosely B, et al., (1987) J Biol Chem 262:2941-2944.
Muhl et al (2003) Eur J Pharmacol 482(325-328)
Murgia M et al (1993) J Biol Chem 268:8199-8203
Parvathenani L K et al (2003) J Biol Chem 278(15):13309-17
Perregaux D G, et al (1992) J Immunol 149:1294-1303.
Perregaux D G and Gabel C A (1994) J Biol Chem 269:15195-15203.
Perregaux D G and Gabel C A (1998) J Immunol 160:2469-2477.
Rutartelli A, et al (1990) EMBO J 9:1503-1510.
Schachter J B et al (1997) Neuropharm 36:1181-1187.
Slater M et al (2004) Histopathology 44(3)206-15
Surprenant et al (1996) Science 272:735-738.
Thornberry N A, et al (1992) Nature 356:768-774.
Trezise D J et al (1994) Eup 3 Pharm 259:295-300.
Vassort G (2001) 81:767-807.
Verhoef P A et al (2003) J Immunol 170:5728-5738.
Virginio C et al (1999) J Physiol 519:335-346.
Wang X (2004) Nat Med 10(8): 821-7
Wang X et al (1999) Moll Cell Cardiol 31:1063-1072.
Wen L T et al (2003) Mol Pharmacol 63:706-713.
Zanovello et al (1990) J Immunol 145:1545-1550.
Zhang X J et al (2004) Leuk Res 28(12):1313-22
Zheng et al (1991) J Cell Biol 112:279-288.
Zoeteweij et al (1996) Hepatology 23:858-865.

1. A method of modulating P2X7 in a patient in need thereof comprising administering a therapeutically effective amount of pyridoxal-5-phosphate or a pharmaceutically acceptable salt thereof to the patient.

2. A method of controlling or mediating inflammation response in a patient in need thereof comprising administering a therapeutically effective amount of pyridoxal-5-phosphate or a pharmaceutically acceptable salt thereof to the patient.

3. A method of decreasing or mediating IL-1β levels in a patient in need thereof comprising administering a therapeutically effective amount of pyridoxal-5-phosphate or a pharmaceutically acceptable salt thereof to the patient.

4. The method according to claim 3, wherein the therapeutically effective amount of pyridoxal-5′-phosphate is between 0.5 and 50 mg/kg body weight.

5. The method according to claim 4, wherein the therapeutically effective amount of pyridoxal-5′-phosphate is between 1 and 15 mg/kg body weight.

6. The method according to claim 3, wherein the patient is human.

7. The method according to claim 3, wherein the patient has a disease or metabolic disorder.

8. The method according to claim 7, wherein the disease or metabolic disorder is selected from the group consisting of: epithelial cancer, leukemia, brain tumor, spinal cord injury, tuberculosis, Alzheimer's Disease, neurodegenerative disease, autosomal recessive polycystic kidney disease, diabetes, prostate cancer, osteoporosis, autoimmune disease, rheumatoid arthritis, multiple sclerosis, myasthenia gravis, Crohn's disease, septic shock, and periodontal infection.

9. The method according to claim 8, wherein the autoimmune disease is selected from lupus erythematosis and rheumatoid arthritis.

10. (canceled)

11. The method of claim 7 wherein the disease or metabolic disorder is an inflammatory disease or disorder.

12. The method of claim 11 wherein the inflammatory disease or disorder is selected from the group consisting of: chronic wound, chronic inflammation, abscess formation, systemic inflammatory response syndrome, including sepsis, appendicitis, arteritis, arthritis, blepharitis, bronchiolitis, bronchitis, bursitis, cervicitis, cholangitis, cholecystitis, chorioamnionitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, gingivitis, hepatitis, hidradenitis suppurativa, ileitis, iritis, laryngitis, mastitis, meningitis, myelitis, myocarditis, myositis, nephritis, omphalitis, oophoritis, orchitis, osteitis, otitis, pancreatitis, parotitis, pericarditis, peritonitis, pharyngitis, pleuritis, phlebitis, pneumonia/pneumonitis, proctitis, prostatitis, rhinitis, salpingitis, sinusitis, stomatitis, synovitis, tendonitis, tonsillitis, uveitis, vaginitis, vasculitis, and vulvitis.

13. The method of claim 7 wherein patient has higher systemtic or local IL-1β levels than a patient without the disease or metabolic disorder.

14. The method of claim 13 wherein the disease or metabolic disorder is selected from the group consisting of: inflammatory bowel disease, ulcerative colitis, Crohn's disease, Sjogren's Syndrome, bone erosion, neuroinflammatory diseases and periodontal disease.

15. The method of claim 14 wherein the neuroinflammatory disease is selected from the group consisting of: Alzheimer's disease, Parkinson's disease, and traumatic brain injury.

16. A method of treating a disease or metabolic disorder selected from the group consisting of: epithelial cancer, leukemia, brain tumor, spinal cord injury, tuberculosis, neurodegenerative disease, autosomal recessive polycystic kidney disease, diabetes, prostate cancer, osteoporosis, autoimmune disease, rheumatoid arthritis, multiple sclerosis, myasthenia gravis, Crohn's disease, septic shock, periodontal infection, chronic wound, chronic inflammation, abscess formation, systemic inflammatory response syndrome, sepsis, appendicitis, arteritis, arthritis, blepharitis, bronchiolitis, bronchitis, bursitis, cervicitis, cholangitis, cholecystitis, chorioamnionitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, gingivitis, hepatitis, hidradenitis suppurativa, ileitis, iritis, laryngitis, mastitis, meningitis, myelitis, myocarditis, myositis, nephritis, omphalitis, oophoritis, orchitis, osteitis, otitis, pancreatitis, parotitis, pericarditis, peritonitis, pharyngitis, pleuritis, phlebitis, pneumonia/pneumonitis, proctitis, prostatitis, rhinitis, salpingitis, sinusitis, stomatitis, synovitis, tendonitis, tonsillitis, uveitis, vaginitis, vasculitis, vulvitis, inflammatory bowel disease, ulcerative colitis, Crohn's disease, Sjogren's Syndrome, bone erosion, neuroinflammatory diseases and periodontal disease in a patient in need thereof, comprising administering a therapeutically effective amount of pyridoxal-5-phosphate or a pharmaceutically acceptable salt thereof to the patient.

17. The method of claim 16 wherein the autoimmune disease is selected from lupus erythematosis and rheumatoid arthritis.

18-22. (canceled)

23. The method of claim 16 wherein the neuroinflammatory disease is selected from the group consisting of: Alzheimer's disease, Parkinson's disease and traumatic brain injury.

24. The method of claim 16, wherein the therapeutically effective amount of pyridoxal-5′-phosphate is about 0.5 to about 50 mg/kg body weight.

25. The use according to claim 24, wherein the therapeutically effective amount of pyridoxal-5′-phosphate is about 1 to about 15 mg/kg body weight.

26. A kit comprising:
a) a therapeutically effective amount of pyridoxal-5-phosphate; and
b) instructions for the administration of the pyridoxal-5-phosphate for the treatment or prevention of a disease or disorder treatable by modulating P2X7.

27. The kit of claim 26 wherein the disease or disorder is selected from the group consisting of: epithelial cancer, leukemia, brain tumor, spinal cord injury, tuberculosis, neurodegenerative disease, autosomal recessive polycystic kidney disease, prostate cancer, osteoporosis, autoimmune disease, rheumatoid arthritis, multiple sclerosis, myasthenia gravis, Crohn's disease, Septic shock, periodontal infection, chronic wound, chronic inflammation, abscess formation, systemic inflammatory response syndrome, including sepsis, appendicitis, arteritis, arthritis, blepharitis, bronchiolitis, bronchitis, bursitis, cervicitis, cholangitis, cholecystitis, chorioamnionitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, gingivitis, hepatitis, hidradenitis suppurativa, ileitis, iritis, laryngitis, mastitis, meningitis, myelitis, myocarditis, myositis, nephritis, omphalitis, oophoritis, orchitis, osteitis, otitis, pancreatitis, parotitis, pericarditis, peritonitis, pharyngitis, pleuritis, phlebitis, pneumonia/pneumonitis, proctitis, prostatitis, rhinitis, salpingitis, sinusitis, stomatitis, synovitis, tendonitis, tonsillitis, uveitis, vaginitis, vasculitis, vulvitis, inflammatory bowel disease, ulcerative colitis, Crohn's disease, Sjogren's Syndrome, bone erosion, neuroinflammatory disease, and periodontal disease.

28. The kit of claim 27 wherein the autoimmune disease is selected from the group consisting of lupus erythematosis and rheumatoid arthritis.

29. (canceled)

30. The kit of claim 27 wherein the neuroinflammatory disease is selected from the group consisting of Alzheimer's disease, Parkinson's disease and traumatic brain injury.

31-34. (canceled)

35. The method according to claim 1, wherein the therapeutically effective amount of pyridoxal-5′-phosphate is about 0.5 to about 50 mg/kg body weight.

36. The method according to claim 2, wherein the therapeutically effective amount of pyridoxal-5′-phosphate is about 1 to about 15 mg/kg body weight.

37. The method according to claim 1, wherein the patient is human.

38. The method according to claim 1, wherein the patient has a disease or metabolic disorder.

39. The method according to claim 5, wherein the disease or metabolic disorder is selected from the group consisting of: epithelial cancer, leukemia, brain tumor, spinal cord injury, tuberculosis, Alzheimer's Disease, neurodegenerative disease, autosomal recessive polycystic kidney disease, prostate cancer, osteoporosis, autoimmune disease, rheumatoid arthritis, multiple sclerosis, myasthenia gravis, Crohn's disease, Septic shock, and periodontal infection.

40. The method according to claim 6, wherein the autoimmune disease is lupus erythematosis or rheumatoid arthritis.

41. The method of claim 5, wherein the disease or metabolic disorder is an inflammatory disease or disorder.

42. The method of claim 8, wherein the inflammatory disease or disorder is selected from the group consisting of: chronic wound, chronic inflammation, abscess formation, systemic inflammatory response syndrome, sepsis, appendicitis, arteritis, arthritis, blepharitis, bronchiolitis, bronchitis, bursitis, cervicitis, cholangitis, cholecystitis, chorioamnionitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, gingivitis, hepatitis, hidradenitis suppurativa, ileitis, iritis, laryngitis, mastitis, meningitis, myelitis, myocarditis, myositis, nephritis, omphalitis, oophoritis, orchitis, osteitis, otitis, pancreatitis, parotitis, pericarditis, peritonitis, pharyngitis, pleuritis, phlebitis, pneumonia/pneumonitis, proctitis, prostatitis, rhinitis, salpingitis, sinusitis, stomatitis, synovitis, tendonitis, tonsillitis, uveitis, vaginitis, vasculitis, and vulvitis.

43. The method of claim 5, wherein the patient has higher systemic or local IL-1β levels than a patient without the disease or metabolic disorder.

44. The method of claim 10, wherein the disease or metabolic disorder is selected from the group consisting of: inflammatory bowel disease, ulcerative colitis, Crohn's disease, Sjogren's Syndrome, bone erosion, neuroinflammatory diseases and periodontal disease.

45. The method of claim 11, wherein the neuroinflammatory disease is selected from the group consisting of: Alzheimer's disease, Parkinson's disease, and traumatic brain injury.

46. The method according to claim 13, wherein the therapeutically effective amount of pyridoxal-5′-phosphate is about 0.5 to about 50 mg/kg body weight.

47. The method according to claim 14, wherein the therapeutically effective amount of pyridoxal-5′-phosphate is about 1 to about 15 mg/kg body weight.

48. The method according to claim 13, wherein the patient is human.

49. The method according to claim 13, wherein the patient has a disease or metabolic disorder.

50. The method according to claim 17, wherein the disease or metabolic disorder is selected from the group consisting of: epithelial cancer, leukemia, brain tumor, spinal cord injury, tuberculosis, Alzheimer's Disease, neurodegenerative disease, autosomal recessive polycystic kidney disease, prostate cancer, osteoporosis, autoimmune disease, rheumatoid arthritis, multiple sclerosis, myasthenia gravis, Crohn's disease, Septic shock, and periodontal infection.

51. The method according to claim 18, wherein the autoimmune disease is lupus erythematosis or rheumatoid arthritis.

52. The method of claim 17, wherein the disease or metabolic disorder is an inflammatory disease or disorder.

53. The method of claim 20, wherein the inflammatory disease or disorder is selected from the group consisting of: chronic wound, chronic inflammation, abscess formation, systemic inflammatory response syndrome, sepsis, appendicitis, arteritis, arthritis, blepharitis, bronchiolitis, bronchitis, bursitis, cervicitis, cholangitis, cholecystitis, chorioamnionitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, gingivitis, hepatitis, hidradenitis suppurativa, ileitis, iritis, laryngitis, mastitis, meningitis, myelitis, myocarditis, myositis, nephritis, omphalitis, oophoritis, orchitis, osteitis, otitis, pancreatitis, parotitis, pericarditis, peritonitis, pharyngitis, pleuritis, phlebitis, pneumonia/pneumonitis, proctitis, prostatitis, rhinitis, salpingitis, sinusitis, stomatitis, synovitis, tendonitis, tonsillitis, uveitis, vaginitis, vasculitis, and vulvitis.

54. The method of claim 17, wherein the patient has higher systemic or local IL-1β levels than a patient without the disease or metabolic disorder.

55. The method of claim 22, wherein the disease or metabolic disorder is selected from the group consisting of: inflammatory bowel disease, ulcerative colitis, Crohn's disease, Sjogren's Syndrome, bone erosion, neuroinflammatory diseases and periodontal disease.

56. The method of claim 23, wherein the neuroinflammatory disease is selected from the group consisting of: Alzheimer's disease, Parkinson's disease, and traumatic brain injury.

57. The method according to claim 37, wherein the patient is human.
